(19) United States
(12) Patent Application Publication
(10) Pub. No.: US 2025/0325779 A1
(43) Pub. Date: Oct. 23, 2025

(54) TRAINING TOOL FOR IMPROVEMENT OF AUTISM SPECTRUM DISORDER

(71) Applicant: CREAMO INC., Seoul (KR)

(72) Inventors: Seok LEE, Seoul (KR); Gang Hyun CHO, Seongnam-si (KR)

(21) Appl. No.: 19/138,039

(22) PCT Filed: Oct. 18, 2023
(86) PCT No.: PCT/KR2023/016114

§ 371 Date: Jun. 11, 2025

(30) Foreign Application Priority Data
Oct. 25, 2022 (KR) 10-2022-0138132

(51) Int. Cl. A61M 21/00; A63H 33/04

(52) U.S. Cl. CPC A61M 21/00; A63H 33/04; A61M 2021/0027; A61M 2021/005; A61M 2205/502; A61M 2205/59

(57) ABSTRACT

The present invention relates to a training tool for the improvement of autism spectrum disorder (ASD), and, more specifically, to a training tool for the improvement of autism spectrum disorder, which is made up of a smart block-shaped teaching aid and accordingly stimulates the interest of a user and allows various emotional expressions to be output according to an input of the user, and is easily linked with software for evaluation and training and thus allows autism spectrum disorder to be easily evaluated, trained, and treated.

### TECHNICAL FIELD

The present invention relates to a training tool for the improvement of autism spectrum disorder (ASD), and, more specifically, to a training tool for the improvement of autism spectrum disorder, which is made up of a smart block-shaped teaching aid and accordingly stimulates the interest of a user and allows various emotional expressions to be output according to an input of the user, and is easily linked with software for evaluation and training and thus allows autism spectrum disorder to be easily evaluated, trained, and treated.

### BACKGROUND ART

Autism spectrum disorder (ASD) is a type of neurodevelopmental disorder that occurs in about 1-2% of the total population, and mainly refers to a neurological syndrome with common characteristics such as difficulty in forming social relationships, problems of emotional interaction, repetitive behavior, and limited interest. The prevalence of such autism spectrum disorder is showing a very high increasing trend worldwide. According to the centers for disease control and prevention (CDC), the diagnosis rate of autism spectrum disorder in the United States increased from 3.57% in 2014 to 4.55% in 2016, and in the case of Korea, as of 2020, the population with autism spectrum disorders is reported to have doubled compared to 2010.

Conventionally, drug or behavioral approach-based treatment means have been proposed as means for the treatment of autism spectrum disorders. However, in the case of behavioral approach-based treatment means, a large part may depend on the experience or technique of the behavioral therapist, and the number of behavioral therapists and treatment institutions are very insufficient, and the reality is that even they are supplied mainly in the metropolitan area, and the current behavioral treatment method is mainly focused on early promotion rather than improvement of problematic behavior. On the other hand, in the case of conventional therapeutic agents such as drugs for treating autism, only two types of Ridpedal and Abilify have been approved for the treatment of autism, but the indications of these drugs are difficult to be regarded as fundamental therapeutic agents due to the lack of efficacy against core symptoms such as social interaction disorders, communication problems, or tendency to engage in repetitive behavior due to the suppression of hyperactivity, and contain fatal side effects of increasing the occurrence of metabolic diseases such as weight gain, hypertension, and diabetes.

In order to overcome the limitations of existing treatment means as described above, digital therapeutics (DTx) have recently been in the spotlight as an alternative. Digital therapeutics is a technology located in the early stages of the world, and there is no clear definition of the concept yet, but according to the Digital Therapeutics Alliance (DTA), a global digital therapeutics industry association formed in 2017, digital therapeutics means a means of “providing evidence-based therapeutic intervention to patients through high-quality software programs that prevent, manage, and treat diseases and disorders,” and according to the Korea Biotechnology Policy Research Center, the concept is defined by describing it as “a new concept of disease prevention, management, and treatment based on digital technology (software) rather than existing eating pills or injections.” Such a digital therapeutic agent has attracted attention as a third-generation therapeutic agent following a first-generation therapeutic agent corresponding to a low-molecular-weight compound such as a pill or a capsule, and a second-generation therapeutic agent which corresponds to a biological agent containing an antibody, a protein, a cell, or the like.

On the other hand, “Mixed Digital Therapeutics” refers to a therapeutic agent that uses software to provide a therapeutic function similar to that of an existing drug, but applies general-purpose hardware (sensors, cameras, VR, etc.) according to the definition of DTA described above. Such a mixed digital therapeutic agent is excellent in portability and accessibility, may dramatically reduce the cost of treatment, has high scalability, is easy to apply to various therapeutic programs, has little side effects, may be used repeatedly for a long period of time, has excellent therapeutic effect compared to a conventional drug/behavioral therapeutic agent due to increased compliance, is very easy to accumulate data, may provide personalized function, and the like, and the effect may be endless. In particular, for developmental disorders characterized by neurological/behavioral symptoms, access by such digital therapeutics is very effective. However, there is currently no hardware-linked hybrid digital therapeutic agent for treating autism spectrum disorder, and only some software-based digital therapeutic agents have been disclosed mainly by advanced foreign companies.

Published Patent Publication No. 10-2017-0046507 (published on May 2, 2017) “Emotional expression robot for counseling and treatment of autistic children”

Although the above-described patent document discloses a method for treating autism using a robot, it has only a configuration in which a very simple form of emoticon is output according to a user's input, and there is a problem in that it is difficult to specifically interact with the user, and it is inevitable to repeatedly perform only simple operations.

### DETAILED DESCRIPTION OF THE INVENTION

### Technical Problem

The present invention has been devised in order to solve the above-described problems.

An object of the present invention is to provide a new smart block-software linked training tool that evaluates whether or not an autism spectrum disorder occurs, and performs training and treatment for the same.

An object of the present invention is to provide a training tool in the form of a mixed-type digital therapeutic agent, which is composed of a teaching tool in a block form, so that a patient with autism spectrum disorder may improve sociality in a natural, easy, and fun way in the process of playing with the teaching tool, and at the same time, improve an individual's ability to express and recognize emotions, thereby improving autism spectrum disorders.

An object of the present invention is to provide a training tool in the form of a block teaching tool capable of easy association with software content capable of evaluating or training for autism spectrum disorder.

An object of the present invention is to provide a training tool that is easy to carry, has no place limitation, is economical as compared with conventional means for treating autism spectrum disorders, and has a high effect of improving autism spectrum disorder by attracting a patient's attention.

An object of the present invention is to provide a training tool the improvement of autism spectrum disorder, which enables more than 70 composite emotional expressions to be recognized and utilized for training, unlike a conventional emotional expression teaching tool capable only of expressing basic emotions.

An object of the present invention is to provide a training tool the improvement of autism spectrum disorder, which is remotely controllable by an administrator such as a parent or a therapist, and enables the datafication, collection, and analysis of various details related to a user's use, thereby making it possible to perform personalized training, treatment, and the like.

On the other hand, other objects not specified in the present invention will be additionally considered within the scope that may be easily inferred from the following problem solving means, the effects of the invention, and the detailed description.

### Solution to Problem

In order to achieve the above object, the present invention is implemented by an embodiment having the following configuration.

According to an embodiment of the present invention, a training tool for the improvement of autism spectrum disorder according to the present invention is a training tool capable of providing training for improvement the disorder in patients with autism spectrum disorder, and includes smart function blocks that are coupled to each other in the form of play blocks and receive power to implement various functions.

According to another embodiment of the present invention, in the training tool for the improvement of autism spectrum disorder according to the present invention, the smart function block includes an input block configured to generate and transmit various input signals; an emotion output block configured to display and output various output signals related to emotions; and a control board configured to transmit and control a control information for the input block and the emotion output block.

According to another embodiment of the present invention, in the training tool for the improvement of autism spectrum disorder according to the present invention, the emotion output block includes a display block configured to output a visual element capable of expressing emotion through an emoticon consisting of a face color and an expression.

According to another embodiment of the present invention, in the training tool for the improvement of autism spectrum disorder according to the present invention, the emotion output block includes an adjacent block recognition unit capable of recognizing an adjacent position of an emotion output block adjacently arranged in a horizontal direction or a vertical direction and an emotion element output from the adjacently arranged emotion output block.

According to another embodiment of the present invention, in the training tool for the improvement of autism spectrum disorder according to the present invention, the emotion output block includes a speaker block configured to output an audible element capable of expressing emotion.

According to still another embodiment of the present invention, in the training tool for the improvement of autism spectrum disorder according to the present invention, the input block, the emotion output block, and the control board each include a body unit formed to have a predetermined space therein so as to form a block having a predetermined volume; an top plate portion coupled to an upper side of the body unit and formed with a protrusion so as to be capable of being coupled to the upper block; and a bottom plate portion coupled to a lower side of the body unit and formed with a coupling groove into which the protrusion is inserted so as to be capable of being coupled to the lower block.

According to another embodiment of the present invention, in the training tool for the improvement of autism spectrum disorder according to the present invention, the training tool further includes a display configured to be linked to the smart function block and outputs various audiovisual contents.

According to another embodiment of the present invention, in the training tool for the improvement of autism spectrum disorder according to the present invention, the training tool further includes a program unit linked to the display and the smart function blocks, and configured to collect, analyze, and manage content output from the display and control information of the smart function blocks.

According to another embodiment of the present invention, in the training tool for the improvement of autism spectrum disorder according to the present invention, the training tool further includes a board on which the smart function block is arranged.

According to another embodiment of the present invention, in the training tool for the improvement of autism spectrum disorder according to the present invention, the board is formed with a projection so as to be capable of being coupled to a lower side of the smart function block.

According to another embodiment of the present invention, a training method for the improvement of autism spectrum disorder according to the present invention includes: a first step of outputting audiovisual content for training; a second step in which a user inputs an emotion expression using the input block in response to the content output in the first step, and a visual and audible output signal corresponding to the emotion expression input from the emotion output block is output and displayed; a third step of arranging the emotion output block on the board by a user; and a fourth step of collecting, determining, comparing, analyzing, and evaluating the content output in the first step and the content related to the emotional expression of the user input, output, and arranged in the second step or the third step, and performing feedback on the training.

According to another embodiment of the present invention, in the training method for the improvement of autism spectrum disorder according to the present invention, the emotion output block includes an adjacent block recognition unit capable of recognizing an adjacent position of an emotion output block adjacently arranged in a horizontal direction or a vertical direction and an emotion element output from the adjacently arranged emotion output block, and the fourth step is configured to collect an adjacent position between the emotion output blocks recognized by the adjacent block recognition unit and an emotion element output from the adjacently arranged emotion output blocks, and determine the collected information as a composite emotion expression.

According to still another embodiment of the present invention, in the training method for the improvement of autism spectrum disorder according to the present invention, the composite emotion includes an amplified emotion, a simultaneous composite emotion, and a time-differentiated composite emotion, and the fourth step may be as follows: when the emotion output block is arranged adjacently in the z-axis direction with respect to the board, it is determined that an emotion element output from the adjacently arranged emotion output block is an amplified emotion; when the emotion output blocks are arranged adjacently in a y-axis direction with reference to the board, it is determined that each of the emotion element output from the adjacently arranged emotions output block is a mixed simultaneous composite emotion; and when the emotions output block is arranged adjacently in the x-axis direction with regard to the board, it is determined that an emotion element is a time-differentiated composite emotion, which means that an emotion changes from an emotion element output from one emotion output block to an emotion element output from the adjacently arranged emotion output block.

### Effects of the Invention

The present invention has the following effects by employing the means for solving the problems disclosed above.

The present invention has the effect of providing a new smart block-software linked training tool that evaluates whether or not an autism spectrum disorder is present, and performs training and treatment for the same.

The present invention has the effect of providing a training tool in the form of a mixed-type digital therapeutic agent, which is composed of a teaching tool in a block form, so that a patient with autism spectrum disorder may improve sociality in a natural, easy, and fun way in the process of playing with the teaching tool, and at the same time, improve an individual's ability to express and recognize emotions, thereby improving autism spectrum disorders.

The present invention has the effect of providing a training tool in the form of a block teaching tool capable of easy association with software content capable of evaluating or training for autism spectrum disorder.

The present invention has the effect of providing a training tool that is easy to carry, has no place restrictions, is economical compared to conventional means for treating autism spectrum disorders, and has a high effect of improving autism spectrum disorder by causing interest of a patient.

The present invention has an effect of providing a training tool for improving autism spectrum disorder, which enables more than 70 composite emotional expressions, unlike a conventional emotional expression teaching tool capable of only expressing a basic emotion, and recognizes and utilizes it for training.

The present invention has an effect of providing a training tool for improving autism spectrum disorder, which is remotely controllable by an administrator such as a parent or a therapist, and enables the datafication, collection, and analysis of various usage details related to the user, thereby making it possible to perform personalized training or therapy.

On the other hand, it goes without saying that effects not explicitly mentioned in the present invention may be treated as described in the present specification as long as they are effects that may be derived within a reasonably inferable range in the description of the entire specification such as the following detailed description.

### BRIEF DESCRIPTION OF DRAWINGS

FIG. 1 is a configuration diagram showing a smart function block according to the present invention.

FIG. 2 is a configuration diagram showing an input block and a display block in a smart function block according to the present invention.

FIG. 3 is a block diagram showing an adjacent block recognition unit in a smart function block according to the present invention.

FIG. 4 is a configuration diagram showing six basic emotions output and displayed in a display block.

FIG. 5 is a configuration diagram showing a detailed coupling relationship of a smart function block according to the present invention.

FIG. 6 is a configuration diagram showing the overall configuration of a training tool according to the present invention.

FIG. 7 is a step diagram showing a training method according to the present invention.

FIG. 8 is a configuration diagram showing a composite emotion expression method according to arrangement of an emotion output block.

### BEST MODE FOR CARRYING OUT THE INVENTION

Preferred embodiments of a training tool for improving autism spectrum disorder according to the present invention will be described in detail below with reference to the accompanying drawings. In the following description of the present invention, in a case where it is determined that a specific description of a known function or configuration may unnecessarily obscure the gist of the present inventions, a detailed description thereof will be omitted. Throughout the specification, when a certain part “comprises” a certain component, it means that other components may be further included without excluding other components unless otherwise specified, and terms such as “unit” and “module” described in the specification mean a unit that processes at least one function or operation, which may be implemented by hardware or software, or a combination of hardware and software.

Unless otherwise defined, all terms (including technical and scientific terms) used herein may be used in a sense commonly understood by one of ordinary skill in the art to which this invention belongs. Also, terms defined in commonly used dictionaries are not to be construed as ideally or unduly unless expressly specifically defined.

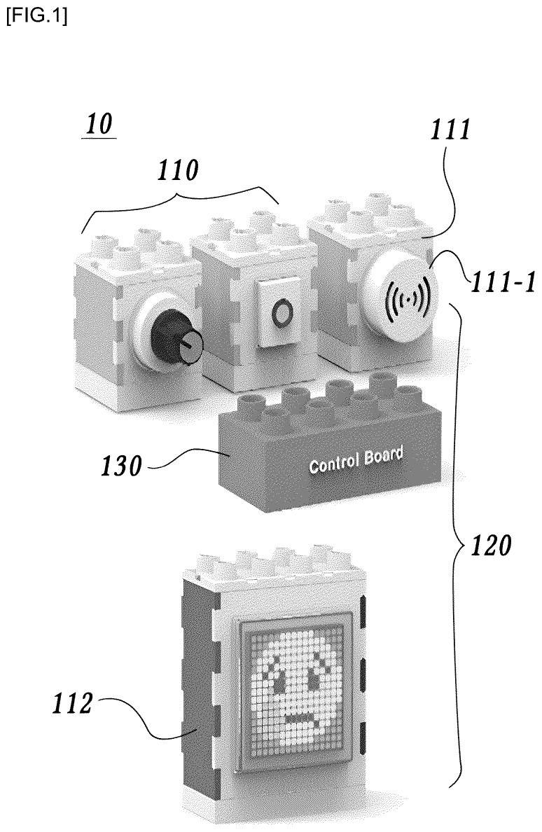

A training tool for the improvement of autism spectrum disorder according to an embodiment of the present invention will be described with reference to FIGS. 1 to 6, wherein the training tool include smart function blocks 10 that are coupled to each other in the form of play blocks and receive power to implement various functions.

The smart function block 10 included in the training tool of the present invention refers to a block in which a smart function is added to a general block that is generally widely used for learning or play, and the smart function refers to a function capable of performing not only a communication function such as IoT but also a function capable of generating and displaying data such as various input/output signals. To this end, the smart function block 10 may include various components such as a communication module and a computation module (CPU), and a hardware configuration including a circuit, a switch, a motor, an LED, a speaker, a sensor, a built-in battery, a charging connector, and the like.

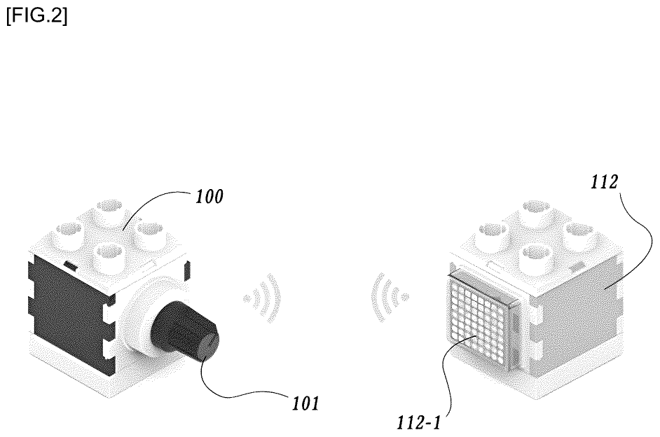

Referring to FIGS. 1 and 2, the smart function block 10 may include an input block 100 configured to generate and transmit various input signals, an emotion output block 110 configured to display and output various output signals related to emotions, and a control board 120 configured to transmit and control a control information for the input block 100 and the emotion output block 110.

The input block 100 is configured such that the smart function block 10 itself generates various input signals and may be linked with other smart function blocks 10, applications, and the like. For example, there may be a switch block that generates a specific signal or a start signal or a stop signal, a dial block that generates an adjustment signal for a specific intensity, or the like. In addition, there may be an on/humidity block, an illuminance block, a camera block that captures and transmits data such as a moving image, or the like that is generated by measuring data such as on/humidities and illuminances. In the present invention, the input block 100 is preferably an input block 100 that is linked with an emotion output block 110 to be described later by a user's operation to output an output signal from the emotion output block 110, and the input block 100 may include, on one surface thereof as shown in FIG. 2, an input switch 101 in the form of a dial that allows the user to input an emotion by directly turning the user's hand, and at the same time allows the user to develop and train small muscles, or an input switch 102 in the form of buttons that allow the user to input the emotion to be output from the linked emotion output block 110 by directly pressing and operating the user.

The emotion output block 110 is configured such that the smart function block 10 itself generates various output signals and may be linked with other smart blocks, applications, and the like. For example, there may be a speaker block that generates a specific sound signal, an LED block that may display specific letters, numbers, and the like, and a motor block that generates a rotational driving force and the like may also be included in the emotion output block 110. As shown in FIG. 1 or FIG. 2, the emotion output block 110 may be provided with a speaker unit 111-1 on one surface of which a specific sound signal is output, or a display unit 112-1 capable of displaying a specific letter, a number, an emoticon, or the like.

The control board 120 is configured to transmit and receive data for controlling in conjunction with the input block 100, the emotion output block 110, and system components such as applications. The control board 120 may be manufactured in the form of a block such as the input block 100 and the emotion output block 110, and may be combined and utilized on a board 20 to be described later and may include various configurations (a communication module, a control module, an interface module, and the like) for interworking with the blocks or system components.

A specific configuration of the emotion output block 110 will be described below. In the training tool according to the present invention, the emotion output block 110 may include a display block 112 configured to output a visual element capable of expressing emotion through an emoticon including a face color and an expression, and a speaker block 111 configured to output various auditory elements capable of expressing emotion. In this case, the emotion output block 110 may include an adjacent block recognition unit 1110 configured to recognize an adjacent position of the emotion output block 110 arranged adjacent to each other in a horizontal direction or a vertical direction and an emotion element output from the adjacent emotion output block 110. The adjacent block recognition unit 1110 may be embedded in the emotion output block 110, so as to be mutually recognizable with the adjacent block recognition unit 1110 embedded in other emotion output blocks 110. For example, one emotion output block 110 may recognize whether another emotion output block 110 arranged adjacent to the block exists through the configuration of the adjacent block recognition unit 1110, and when the adjacent emotion output block 110 exists, it may recognize whether the adjacent emotion output blocks 110 are adjacent in a horizontal direction or a vertical direction, and in the case of a horizontal direction, it may recognize a relative adjacent position in a plane such as an x-axis or a y-axis. In addition, when the adjacent block recognition unit 1110 recognizes whether another emotion output block 110 is adjacently arranged, an arrangement position, and the like, it shares and transmits a control command performed in the adjacently arranged emotion output block 110, so that an emotion input to the adjacently arranged emotion output block 110 or an emotion element output from the adjacently arranged emotion output block 110 may be recognized. The configuration of the adjacent block recognition unit 1110 communicates with and interworks with the control board 120 or a program unit (not shown) to be described later, so as to recognize a composite emotional expression of a user and provide a training program or feedback that may be associated therewith. This will be described later. On the other hand, the meaning of “adjacent” preferably means that the blocks are arranged side by side on the board 20 in the horizontal direction, and preferably means that the block is arranged side by side in the vertical direction with respect to the board 20 in a vertical direction, but it may be treated that other block components having a thickness of 2 cm or less, which are additionally configured, are included in “adjacent,” even if they are arranged between the emotion output blocks 110 arranged adjacent to each other.

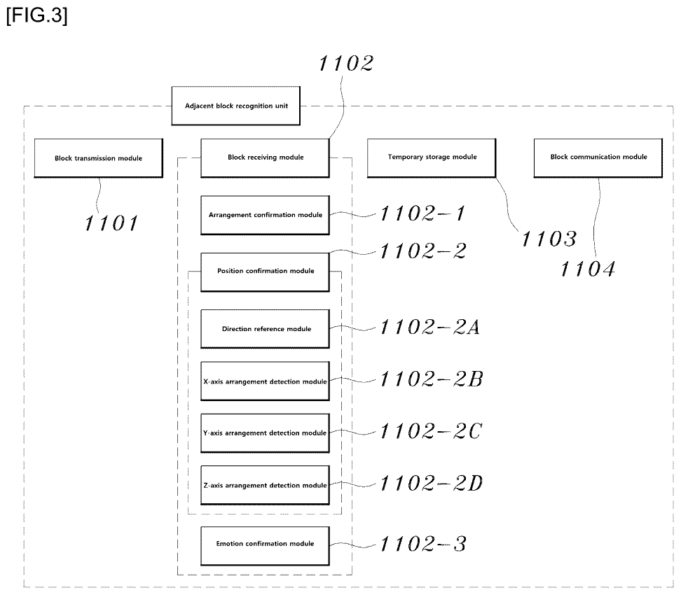

Referring to FIG. 3, the adjacent block recognition unit 1110 may include a block transmission module 1101 configured to transmit data such as unique information of a block itself, whether an emotion is input/output from a user, and a type of the input/output emotion in short-range communication at regular intervals, a block reception module 1102 configured to receive and sense matters transmitted from other emotion output blocks 110, a temporary storage module 1103 configured to temporarily store data such as an arrangement of the other emotion output block 110 and input/output emotion, a transmission/reception time, and the like, and a block communication module 1104 configured to transmit and exchange details stored in the temporary storage module 1103, to a program unit (not shown) or an external system to be described later.

The block transmission module 1101 is configured to transmit data such as unique information of a block itself, whether an emotion is input/output from a user, and a type of the input/output emotion in short-range communication at regular intervals, and may mean a configuration in which short-range communication is performed so that other emotion output block 110 recognizes the presence of the emotion output block 110, and any configuration is not particularly limited as long as the short-range communication corresponds to a known method such as nRF or Bluetooth.

The block reception module 1102 is configured to receive and sense the details transmitted from the block transmission module 1101 of the other emotion output block 110 to obtain the data of whether the other emotion output blocks 110 are arranged adjacently, the position of the adjacent arrangement, the type of the input/output emotion, and the like, and for this purpose, the block reception module 1102, may include an arrangement confirmation module 1102-1 for confirming whether the other emotion input blocks 110 are arranged contiguously, a position confirmation module 1102-2 for confirming the position of the arrangement when the arrangement of the other emotion input block 110 is confirmed adjacently by the arrangement confirmation module 1102-1, and an emotion confirmation module 1102-3 for confirming the emotion element input/output to the emotion output block 110 from the transmission content when the arrangement of other emotion output block 110 is confirmed contiguously by the arrangement confirmation module 1102-1.

The arrangement confirmation module 1102-1 is configured to, when a signal transmitted from the block transmission module 1101 of the other emotion output block 110 is received and sensed, confirm whether the other emotion output blocks 110 are arranged adjacent to the emotion output block 110 through whether the signal is sensed with a sensitivity greater than or equal to a reference value.

The position confirmation module 1102-2 is configured to confirm the arrangement position when adjacent placement of other emotion output block 110 is confirmed by the arrangement confirmation module 1102-1, and to this end, the position confirmation module 1102-2 may include a direction reference module 1102-2A configured to set a reference direction in three-dimensional space; an x-axis arrangement detection module 1102-2B configured to confirm whether adjacent arrangement has occurred along the x-axis based on the reference set by the direction reference module 1102-2A with respect to the placement of the board 20; a y-axis arrangement detection module 1102-2C configured to confirm whether adjacent arrangement has occurred along the y-axis based on the reference set by the direction reference module 1102-2A; and a z-axis arrangement detection module 1102-2D configured to confirm whether adjacent arrangement has occurred along the z-axis based on the reference set by the direction reference module 1102-2A.

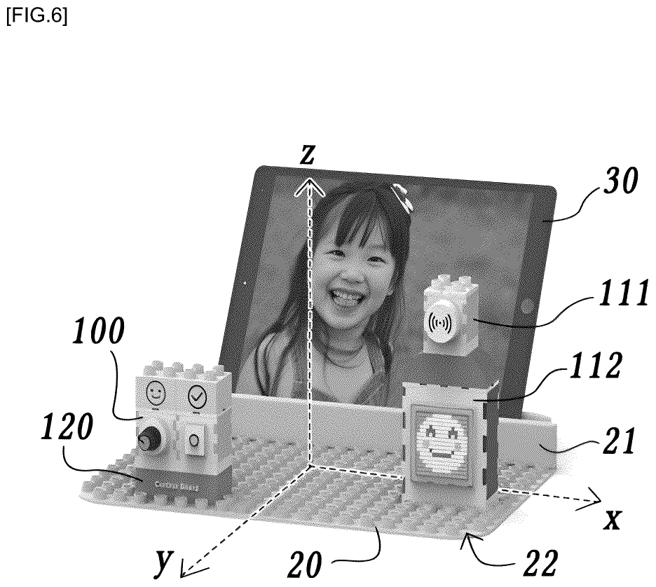

The direction reference module 1102-2A is configured to set a direction reference at an arrangement position, and the user may optionally use an appropriate reference as the direction reference, and in the training tool of the present invention, the direction reference is preferably configured to recognize a direction in which the speaker unit 111-1 or the display unit 112-1 formed on one surface of the emotion output block 110 is formed in the y-axis direction, and set a three-dimensional direction reference based on this. Therefore, according to the above criteria, as shown in FIG. 6, when the speaker unit 111-1 or the display unit 112-1 is arranged side by side with each other or arranged side by side to the rear surface thereof, it may be regarded as being arranged in the x-axis direction, and when it is arranged in the horizontal direction while being vertical with respect to the x-axis directions, it may be viewed as being arranged in a y-axis direction. This directional reference is shown in FIG. 6.

The temporary storage module 1103 may be configured to temporarily store, when the adjacent arrangement of the other emotion output blocks is recognized, time information such as unique information of the recognized other emotion output block 110, whether the other emotion output block is adjacently arranged, an adjacent arrangement position, data related to an emotion input/output from the recognized other emotion input block 110, and a recognition time.

The block communication module 1104 is configured to transmit and exchange the information stored in the temporary storage module 1103 to a program unit (not shown) or an external system to be described later, and based on this, the program unit (not shown) or the external system may recognize an adjacent arrangement of the emotion output block 110 and detect a composite emotion expression corresponding thereto.

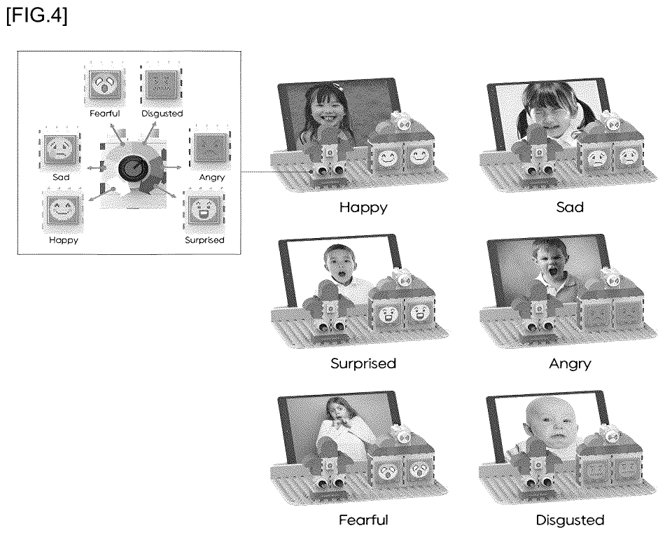

Referring to FIG. 4, the display block 112 includes a display unit 112-1 capable of outputting a visual element on one surface, and may output a visual element capable of expressing an emotion through an emoticon including a face color and an expression in conjunction with the input block 100. In the present invention, the emoticon preferably means an emoticon in the form of expressing six basic emotions of joy, sadness, fear, dislike, anger, and surprise as shown in FIG. 4, but does not necessarily mean only an emoticon of the form as shown in FIG, 4, and the type and number of each emotion may be appropriately selected and adopted by a person skilled in the art.

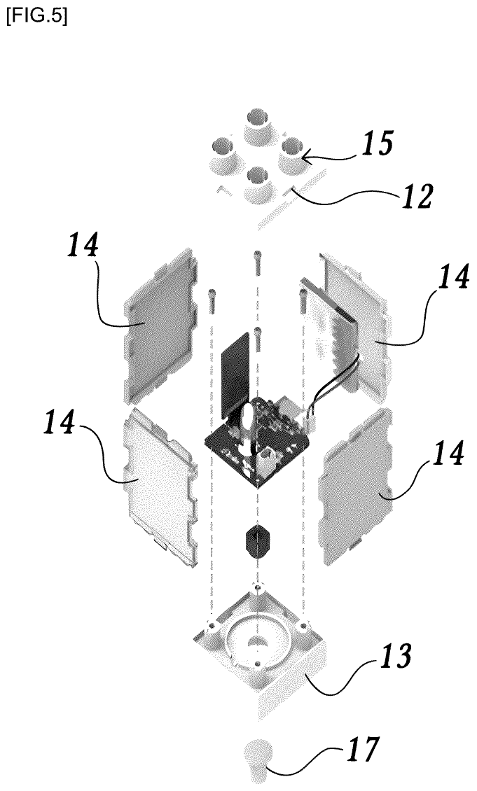

Hereinafter, specific configurations of the input block 100, the emotion output block 110, and the control board 120 of the training tool according to the present invention will be described with reference to FIG. 5. The input block 100, the emotion output block 110, and the control board 120 of the training tool according to the present invention may include a body unit 11 formed to have a predetermined space therein so as to form a block having a predetermined volume, an top plate unit 12 coupled to an upper side of the body unit 11 and formed with a protrusion 15 to be coupled to the upper block, and a bottom plate unit 13 coupled to a lower side of the body unit 11 and formed with a coupling groove 16 into which the protrusion 15 is inserted to be coupled to a lower block. At this time, the top plate unit 12 and the bottom plate unit 13 may include protrusions 15 and coupling grooves 16 that are compatible with commercially available block products such as LEGO and Oxford and are respectively coupled and detached, so that the top plate unit 12 and the bottom plate unit 13 of the smart function block 10 may be coupled to each other, and furthermore, the upper and lower surfaces of the board 20 may be configured to be detachable from each other with respect to the upper and lower surface of the commercially available general block product as described above.

In addition to the general detachable function, the top plate unit 12 or the bottom plate unit 13 may be configured such that a power line or the like is additionally formed therein to receive power from the board 20 or to enable power transmission/reception or data communication between the smart function blocks 10. On the other hand, in the case of the bottom plate unit 13, a separate lower switch 17 may be further formed, and when the lower switch 17 is mounted and coupled to the protrusion 22 formed on the upper surface of the block or the board 20, the lower switch 17 may physically pressurize to perform electrical connection, so that the switch is converted to the ON mode in the form of PUSH to start operation, and when detached and released, the electrical connection is released while returning to the original state, so that the electrical connection is converted to the OFF mode to stop operation.

A hardware component for implementing various functions of the smart function block 10 may be additionally included in the body unit 11.

For example, a communication module for performing communication of the smart function block 10 may be configured, and the communication module may mean that a Global License-Free 2.4 GHz ISM band is used as a wireless communication board and independent FSK modulation suitable for a smart toy application is used.

Alternatively, a battery for supplying power to the smart function block 10 may be configured, or an external charging unit may be further configured. The battery may refer to a Li-ion/Polymer battery, and the external charging unit may utilize various general-purpose terminals such as a Micro USB, and a dedicated charging control circuit may be further formed.

Alternatively, an operation module responsible for performing the overall function of the smart function block 10 may be configured, and the operation module may include various types of main boards (common, LED, motor, etc.) according to functions as a main board, and may further include an open hardware-based main control circuit and a connection unit for function scalability.

Alternatively, an additional module responsible for performing an additional function of the smart function block 10 may be configured, and an example of the additional module may refer to the adjacent block recognition unit 1110 as described above.

It goes without saying that the above-described hardware components may include all or only some of the respective components, and as an example, functional components other than the above-described configurations may be included.

The body unit 11 may be formed with a plate 14 partitioning the body unit 11 from the outside to form a block of a certain volume, and the plate 14 may be formed to be suitable for various sensors, displays, and UIs according to the functionality of the smart function block 10, and may be formed using a harmless and safe material in consideration of a child who is a main user.

An embodiment including an additional configuration of a training tool according to the present invention will be described with reference to FIG. 6. The training tool for improving autism spectrum disorder according to the present invention may further include a display 30 that is interoperable with the smart function block 10 and outputs various audio-visual contents, and may further include a program unit (not shown) that is interoperatable with the display 30 and the smart function block 10 and collects, analyzes, and manages contents output from the display 30 and control information of the smart function block.

The display 30 may mean any means capable of outputting audiovisual content for autistic disorders such as autism spectrum syndrome, further cognitive or behavioral disorders improvement, and training. The display 30 is preferably capable of outputting the content under the control of the program unit (not shown), but may be configured to output the content embedded in the display 30 by operating the display 30 itself without the control of the programs unit (not shown) as necessary. The display 30 may refer to a display that may be separately manufactured or provided, or may refer to utilizing an existing smart product capable of outputting audio-visual content, such as a smart pad of a smartphone, a PDA, an iPad, or the like.

The program unit (not shown) may refer to a means for collecting, analyzing, and managing the content output from the display 30 and the control information of the smart function block 10 in association with the display 30 and smart function block 10, and the program unit (not shown) may be mounted on or driven by a separate external system, or may be mounted on and driven by a PC of an administrator in a form of a computer program or the like, or may be mounting on and driven by the mobile of an administrator in the form of an application or the like. The program unit (not shown) may store and manage audio-visual content output from the display 30 in conjunction with the display 30, and may control matters such as content driving, volume, brightness, and pause of the display 30. On the other hand, the program unit (not shown) may collect control information of the smart function block 10 operated by the user in response to content in conjunction with the smart function block, and may analyze the collected control information to observe a pattern, identify a training pattern, and provide personalized feedback. A description of a training method according to a configuration of such a program unit (not shown) will be described later.

Still referring to FIG. 6, the training tool may further include a board 20 on which the smart function block 10 is arranged, a protrusion 22 may be formed on an upper surface of the board 20 so as to be capable of being coupled to a lower side of the smart function block, and a coupling groove 23 may be formed on a lower surface so as to be capable of being coupled to an upper side of the smart functional block 10. The protrusions 22 and the coupling grooves 23 may be formed in the same configuration as the configuration formed in the smart function block 10, so as to be compatible with commercially available block products such as LEGO, Oxford, and the like, and to be detachably coupled to each other. In addition, the board 20 may further include a support unit 21 capable of supporting the display 30.

Meanwhile, the board 20 may mean a smart board having a smart function. While the smart function block 10 is arranged, the smart board recognizes the arranged smart function block 10, and implements various learning, play, games, and the like in conjunction with the smart function block and the application, and basically implements various functions such as position recognition (Plug & Play method, etc.), power supply (charging, etc.), and transmission/reception of various input/output signals or control signals for the smart function block. In particular, the smart board may be configured to allow the user to customize and relocate the area of the board 20, thereby maximizing the usability of the smart board of a limited size and allowing the user to use the smart board in various ways.

The smart function block 10 of the training tool according to the present invention is assumed to have a structure in which a battery is built in and power may be supplied by the block itself, but in contrast, a power block (not shown) in which a separate battery is not built in, a power line is formed in the bottom plate unit 13 or the top plate unit 12, and power is separately transmitted to the smart function block 10 may be configured to supply power by arranging the power block (not shown) adjacent to the smart function blocks 10 and connecting the power lines. In addition, through the configuration of a beacon block (not shown) in which a beacon module is separately built in and reference position information is transmitted wirelessly, the beacon block is arranged adjacent to the smart function block 10 and a line is connected, so that the reference position information may be transmitted to the surrounding smart function block 10, a control device, a system, and the like.

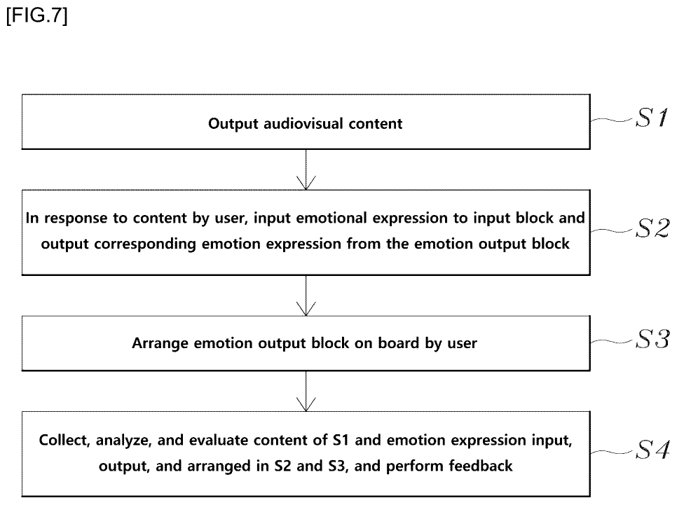

Hereinafter, a training method employing a training tool for improving autism spectrum disorder according to the present invention will be described with reference to FIG. 7. In the following, detailed descriptions of components common to those described in the foregoing embodiments are omitted.

As disclosed in FIG. 7, the training method is a training method using the training tool according to the present invention, and includes a first step S1 of outputting audiovisual content for training, a second step S2 in which a user inputs an emotion expression using the input block 100 in response to the content output in the first step S1, and a visual and audible output signal corresponding to the emotion expression input from the emotion output block 110 is output and displayed, a third step S3 in which arranging the emotion output block 100 on the board 20 by the user, and a fourth step S4 of collecting, determining, comparing, analyzing, and evaluating the content output in the first step S1 and the content related to the emotional expression of the user input, output, and arranged in the second step S2 or the third step S3, and performing feedback on the training.

The first step S1 may refer to a step of outputting audiovisual content for training on the display 30. The content may be manipulated and reproduced by an administrator through the program unit (not shown), or may be automatically reproduced according to a preset process in software for a training program. The training audiovisual content may refer to audiovisual content for improving and training an autistic disorder including an autism spectrum disorder, and further a cognitive disorder or a behavioral disorder, and the audiovisual content includes content sequentially displayed with a certain time difference according to a type of emotion representing a face of a person expressing an emotion, and the person may refer to a person of various ages, races, genders, or the like, and may be displayed using a face of a user's parent, brother, friend, or the like.

In the second step S2, the user responding to the content operates the input block 100 to input his/her own emotional expression, and an emotional expression corresponding to the emotional expression input to the input block 100 is output and displayed on the emotion output block 110. For example, when a facial expression of a person making a happy expression is displayed in the content, the user may view the facial expression and input a specific emotional expression by operating the input block 100, and a corresponding emotional expression may be output and displayed in the emotion output block 110 according to the input expression. In this case, if the user manipulates the input block 100 to output an emoticon and a voice corresponding to “pleasure” and an output corresponding thereto is made in the emotion output block 110, the manager or training program may give a higher evaluation of the user's emotion recognition or sympathetic ability, and if the emoticon and the voice corresponding to “angry” are to be output, an otherwise evaluation may be given.

The third step S3 may refer to a step of arranging the emotion output block 110 on the board 20 after the input operation performed by the user in the second step S2. At this time, the user may express a composite emotion by arranging a plurality of the emotion output blocks 110 on the board 20 in a constant arrangement.

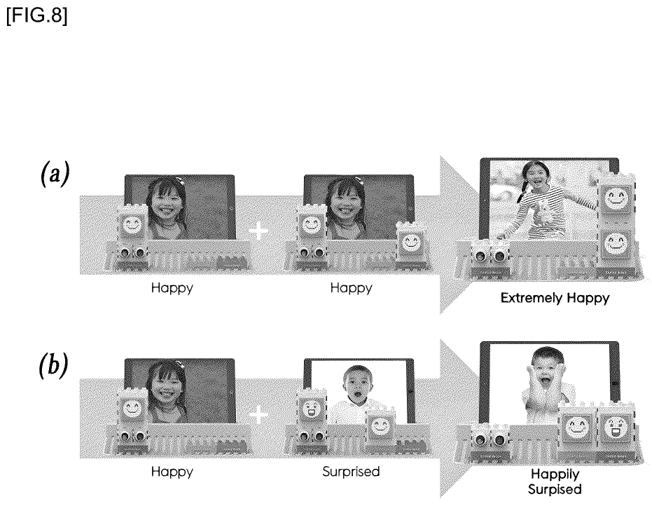

With reference to FIG. 8, the method for expressing composite emotions will be described in more detail. With the configuration of the adjacent block recognition unit 1110, the emotion output block 110 may recognize an adjacent position, an emotion element, and the like of the adjacently arranged emotion output block 110, which may be collected, analyzed, and determined by a program unit (not shown) or an external system, and a method of setting a position reference such as an x-axis, a y-axis, and a z-axis are as described above. The user may use this to express a composite emotion, including an amplified emotion, a simultaneous composite emotion, and a time-differentiated composite emotion. The user may express an “amplified emotion” by arranging the emotion output block 110 adjacent in the x-axis direction on the horizontal with respect to the board 20, may express a “simultaneous composite emotion” by adjoining the emotion output blocks 110 in the y-axis direction on a horizontal with respect to a board 20, and may express a “time-differentiated composite emotion” by adjacently arranging an emotion output block 110 in the vertical direction, that is, in the z-axis direction with respect to aboard 20.

As shown in FIG. 8A, the user may express an amplified emotion. An amplified emotion herein may mean a “relatively amplified base emotion.” For example, according to a preferred embodiment of the present invention, the user may express six basic emotions of joy, sadness, fear, dislike, anger, and surprise by using the emotion output block 110, and the amplified emotion expression may express a composite emotion of very joy, very sadness, very fear, very dislike, very anger, and very surprise. As in the example illustrated in FIG. 8A, the user may express an amplified emotion of “very joy” by arranging two emotion output blocks 110 that output “joy” adjacent to each other in a vertical direction, that is, in a z-axis direction with respect to the board 20.

As shown in FIG. 8B, the user may express a time-differentiated composite emotion. In the present specification, the time-differentiated composite emotion may mean “an emotion change over time or a composite emotion accompanied therewith.” For example, according to a preferred embodiment of the present disclosure, the user may express six basic emotions of joy, sadness, fear, dislike, anger, and surprise by using the emotion output block 110, and may express 30 composite emotions combining the basic emotions such as sadness after joy, fear after joy, dislike after joy, and the like by using the time-differentiated composite emotion expression. As in the example illustrated in FIG. 8B, the user may express a time-differentiated composite emotion of “grief after joy” by arranging the emotion output block 110 that outputs “joy” and the emotion output block 100 that outputs “surprise” adjacent to each other in the x-axis direction with respect to the board 20.

On the other hand, although not shown separately in the drawings, the user may express a simultaneous composite emotion. Simultaneous composite emotions herein may mean “composite emotions in a state in which basic emotions are combined.” For example, according to a preferred embodiment of the present disclosure, the user may express six basic emotions of joy, sadness, fear, dislike, anger, and surprise by using the emotion output block 110, and may express 30 composite emotions combining the basic emotions, such as joy and sadness, joy and fear, joy and dislike, and the like by simultaneously expressing composite emotions. For example, the user may express a time-differentiated composite emotion of “pleasure and sadness” by arranging the emotion output block 110 that outputs “pleasure” and the emotion output block 100 that outputs “surprise” adjacent to each other in the y-axis direction with respect to the board 20.

The fourth step S4 may refer to a step of collecting, determining, comparing, analyzing, and evaluating the content output in the first step S1 and the emotional expression of the user input, output, and arranged in the second step S2 or the third step S3, and performing feedback on training. The training tool according to the present invention may collect and manage information, location information, and the like related to the operation and control of the content and the control performed on the input block 100 or the emotion output block 110 in the program unit (not shown) or an external system, and may evaluate this by a separate training program (human resources or software). Therefore, it is possible to perform functions such as analyzing and evaluating cognitive ability/sympathetic ability and the like based on the user's response to the displayed content, configuring and managing a personalization program based on this, providing feedback tailored to the user's cognitive ability/sympathy ability, and adjusting the difficulty of the training program.

In the fourth step S4, according to the arrangement of the emotion output block 110 by the user in the third step S3, the composite emotion expression may be recognized, collected, and used as a determination element. The adjacent block recognition unit 1110 may transmit the temporarily stored data to a program unit (not shown) or an external system according to the configuration of the block communication module 1104, and based on this, may recognize a composite emotional expression according to the adjacent arrangement of the emotion output block 110. Based on this, the emotional expression of the user may be compared, analyzed, and evaluated, and feedback on training may be performed as described above.

In the foregoing, the Applicant has described various embodiments of the present invention, but such embodiments are only one embodiment of implementing the technical idea of the present invention and any change or modification shall be construed as falling within the scope of the present invention as long as the technical idea of this invention is implemented.

1. A training tool for the improvement of autism spectrum disorder, capable of providing training for improvement the disorder in patients with autism spectrum disorder, comprising:
smart function blocks that are coupled to each other in the form of play blocks and receive power to implement various functions.

2. The training tool for the improvement of autism spectrum disorder of claim 1, wherein the smart function block comprises:
an input block configured to generate and transmit various input signals; an emotion output block configured to display and output various output signals related to emotions; and a control board configured to transmit and control a control information for the input block and the emotion output block.

3. The training tool for the improvement of autism spectrum disorder of claim 2, wherein the emotion output block comprises:
a display block configured to output a visual element capable of expressing emotion through an emoticon consisting of a face color and an expression.

4. The training tool for the improvement of autism spectrum disorder of claim 2, wherein the emotion output block comprises:
an adjacent block recognition unit capable of recognizing an adjacent position of an emotion output block adjacently arranged in a horizontal direction or a vertical direction and an emotion element output from the adjacently arranged emotion output block.

5. The training tool for the improvement of autism spectrum disorder of claim 2, wherein the emotion output block comprises:
a speaker block configured to output an audible element capable of expressing emotion.

6. The training tool for the improvement of autism spectrum disorder of claim 2, wherein the input block, the emotion output block, and the control board each comprise:
a body unit formed to have a predetermined space therein so as to form a block having a predetermined volume; an top plate portion coupled to an upper side of the body unit and formed with a protrusion so as to be capable of being coupled to the upper block; and a bottom plate portion coupled to a lower side of the body unit and formed with a coupling groove into which the protrusion is inserted so as to be capable of being coupled to the lower block.

7. The training tool for the improvement of autism spectrum disorder of claim 1, wherein the training tool further comprises:
a display configured to be linked to the smart function block and outputs various audiovisual contents.

8. The training tool for the improvement of autism spectrum disorder of claim 7, wherein the training tool further comprises:
a program unit linked to the display and the smart function blocks, and configured to collect, analyze, and manage content output from the display and control information of the smart function blocks.

9. The training tool for the improvement of autism spectrum disorder of claim 1, wherein the training tool further comprises:
a board on which the smart function block is arranged.

10. The training tool for the improvement of autism spectrum disorder of claim 9, wherein the board is formed with a projection so as to be capable of being coupled to a lower side of the smart function block.

11. A training method for the improvement of autism spectrum disorder using the training tool of claim 1, comprising:
a first step of outputting audiovisual content for training;
a second step in which a user inputs an emotion expression using the input block in response to the content output in the first step, and a visual and audible output signal corresponding to the emotion expression input from the emotion output block is output and displayed;
a third step of arranging the emotion output block on the board by a user; and
a fourth step of collecting, determining, comparing, analyzing, and evaluating the content output in the first step and the content related to the emotional expression of the user input, output, and arranged in the second step or the third step, and performing feedback on the training.

12. The training method for the improvement of autism spectrum disorder of claim 11, wherein the emotion output block comprises an adjacent block recognition unit capable of recognizing an adjacent position of an emotion output block adjacently arranged in a horizontal direction or a vertical direction and an emotion element output from the adjacently arranged emotion output block, and the fourth step is configured to collect an adjacent position between the emotion output blocks recognized by the adjacent block recognition unit and an emotion element output from the adjacently arranged emotion output blocks, and determine the collected information as a composite emotion expression.

13. The training method for the improvement of autism spectrum disorder of claim 12, wherein the composite emotion comprises:
an amplified emotion, a simultaneous composite emotion, and a time-differentiated composite emotion, and
the fourth step may be as follows: when the emotion output block is arranged adjacently in the z-axis direction with respect to the board, it is determined that an emotion element output from the adjacently arranged emotion output block is an amplified emotion; when the emotion output blocks are arranged adjacently in a y-axis direction with reference to the board, it is determined that each of the emotion element output from the adjacently arranged emotions output block is a mixed simultaneous composite emotion; and when the emotions output block is arranged adjacently in the x-axis direction with regard to the board, it is determined that an emotion element is a time-differentiated composite emotion, which means that an emotion changes from an emotion element output from one emotion output block to an emotion element output from the adjacently arranged emotion output block.
